(19) United States
(12) Patent Application Publication
(10) Pub. No.: US 2022/0280493 A1
(43) Pub. Date: Sep. 8, 2022

(54) TREATMENT OF A RESPIRATORY CORONAVIRUS INFECTION WITH NITROXIDES

(71) Applicant: Peter Herbert Proctor, Houston, TX (US)

(72) Inventor: Peter Herbert Proctor, Houston, TX (US)

(21) Appl. No.: 17/678,990

(22) Filed: Feb. 23, 2022

Related U.S. Application Data
(60) Provisional application No. 63/158,122, filed on Mar. 8, 2021.

(51) Int. Cl. A61K 31/445; A61K 31/4468; A61K 31/45; A61K 31/15; A61K 31/04; A61K 31/675

(52) U.S. Cl. CPC A61K 31/445; A61K 31/4468; A61K 31/45; A61K 31/15; A61K 31/04; A61K 31/675

(57) ABSTRACT

The use of nitroxide and nitrone compounds for the treatment of acute, breakthrough, or “long-haul” respiratory coronavirus infections is claimed. Exemplars include Tempol and its reduced prodrug hydroxylamine form as well as PBN (N-Benzylidene-tert-butylamine N-oxide). Methods of administration include nasal insufflation, inhalation, topical to skin or mucosa, orally, intradermally, subcutaneously, by intravenus infusion, using suitable pharmacologically-active formulations.

Priority is claimed to Provisional Pat. No. 63/158,122, dated Mar. 8, 2021

Not Applicable

Not Applicable

### BACKGROUND

### Prior Art

This patent relates to the treatment of a respiratory coronavirus, (especially SARS-COV-2) infection with a nitroxide drug. The previously-unknown disease (“Covid-19”) caused by this virus has recently resulted in a massive world-wide epidemic with millions dead and a partial shutdown of the world economy. Post-acute, “Long-Haul” Covid-19 is also a significant cause of morbidity and mortality. Thus, there is an urgent need for an orally-effective, non-toxic, and relatively inexpensive agent to treat Covid-19, both acutely, in “break-thru” disease and in its long-haul manifestation. Likewise, with respect to the treatment of Covid-19, “The availability of medications with different mechanisms of action offers the opportunity for creating combination therapies that are potentially synergistic and less likely to lead to resistance.”

Oxidative processes, modulated both by direct chemical attack and by modulation of biological processes by redox processes or “Redox Signaling” have long been known to play a key role in the etiology of inflammatory diseases, cancer, and degenerative diseases (Proctor, 1989). This includes lung diseases such as pulmonary fibrosis and ARDS. Examples of such redox-effector molecules include superoxide free radical, hydrogen peroxide, their daughter products, and the like.

For example, superoxide dismutase enzymes (“SOD's”) catalyze the dismutation of superoxide free radicals to peroxide and water. Such SOD's, as well as small-molecule SOD-mimetic nitroxide spin labels such as Tempol, have antiinflammatory activity in a variety of human and animal disease. Other, more specific, actions are may be connected to the action of superoxide and its daughter products as cellular messengers. Direct effects of the molecules themselves may also figure. One example is modulation of the hair cycle, another is cancer cell growth and metastasis. Similarly, nitroxides may paradoxically act as pro-oxidants, as well as antioxidants. For example, such compounds may mimic the action of the naturally-occuring mediator nitric oxide (NO), Proctor, (1986). For reviews, see Proctor P (1989), Wilcox C S. (2010), Zarling J A, et al (2015), and Lewandowski M and Gwozdzinski K. (2017), which are incorporated by reference. Thus, in the mid 1980's, the applicant first discovered the pharmaceutical actions of nitroxide spin labels and spin traps. Applicant subsequently patented several such compounds for certain applications related to oxidative stress in humans (U.S. Pat. No. 5,728,714A). Similarly, applicant has previously informally used Tempol to treat colds (many of which involve a coronavirus), as well as influenza. E.g., applicant's U.S. Pat. No. 8,778,969B2, notes (but does not claim) the use of Tempol to treat certain viral infections, including influenza. While undergoing continual development and refinement by the applicant, the use of Tempol in such self-limited viral infections has not been otherwise formalized. Nor, while continuing to develop and further refine it, has applicant ever otherwise noted this utility previous to Proctor, 2020. (Free Rad. Biol. Med. 2020 Nov. 1; 159: S95)

Thus, the final ‘969 patent only claims the use of Tempol and allied compounds for the treatment of fibrocystic disease of breast. Unfortunately, this cancer-related utility has also not been commercialized. Similarly, although the condition of “Radiologically-Dense Breasts” is the single most important clinical precurser for breast cancer, our patent U.S. Pat. No. 10,028,943 (claiming treatment of this condition with Tempol and its sister nitroxides), has also not been picked up for further development. Similar discoveries from (e.g.) the National Cancer Institute claiming the use of tempol or its derivatives to treat, e.g., various cancers have also not been commercialized (for reviews, see, Wilcox C S. (2010), and Zarling J A, et al (2015), and Lewandowski M and Gwozdzinski K. (2017)). Likewise, the one attempt by a large drug company to commercialize the nitrone disulfonyl-PBN (NXY-059) for the treatment of acute ischemic stroke ended up a last-minute failure (Proctor and Tamborello, 2007). This wiped several billion dollars off the net asset value of the sponsoring drug company, AstraZenica.

More broadly, because of this latter experience and the general failure of promising results in animal studies to replicate in humans, for a time major drug companies were simply uninterested in any antioxidant drug, much less a nitrone or nitroxide. Stated-simply, positive results with antioxidant drugs in animal studies have not translated to humans. This argues strongly against “obviousness” based upon results with such drugs in animal models. Thus, Traystman, R J (2010) teaches with respect to antioxidant neuroprotective drugs, “There are hundreds, perhaps thousands of neuroprotective drugs that have been used in animal models. So, if you were a mouse or a rat, and experienced a stroke or cardiac arrest, we would know just what to do for you. But, essentially none of these pharmacological agents have demonstrated usefulness in humans even though they have been shown to be successful in preclinical animal trials”.

Similarly, U.S. Pat. No. 10,441,568B2 claims the use of certain 5-membered-ring cyclic nitroxides (but not Tempol or its derivatives) for the treatment of disorders of the respiratory tract of experimental animals. Similarly, Tsuhako et al (2010) report that Tempol ameliorates murine viral encephalomyelitis induced by a nonrespiratory coronavirus unrelated to SARS-CoV-2. Likewise, applicant's pending patent application Ser. No. 17/354,068 (incorporated by reference) claims use of Tempol and allied compounds for the treatment of SARS-COV-2 infection per se.

Similarly, the speed with which Tempol works in human Covid-19, implies that Tempol may be directly antiviral, Such direct antiviral action is contrary to the standard model of Tempol as an in vivo antioxidant and SOD-mimetic. However, it is in accord with our original model postulating that such nitroxide drugs may act as NO analogs (Proctor, 1986). Arguably, both these and additional mechanisms are in play, making Tempol a truly unique drug with multiple mechanisms of action. Nor are our claims dependent upon any specific mechanism of action or theory for validity. Similarly, diseases are very complicated. Thus, efficacy in one specific symptom or sign in one distinct disease does not confer “obviousness” that a drug will work in another. Likewise, the usual finding with antioxidant drugs is that results in preclinical studies do not translate to humans. This is especially the case where a particular disease was completely unknown until recently.

### BRIEF SUMMARY OF INVENTION

The present invention is directed to methods that treat, inhibit, or slow the development of the symptoms of respiratory coronaviruses. This particularly includes SARS-CoV-2 infection (“Covid-19”) in its “Long-Haul” and break-through manifestations, both acutely and longer term. Among other symptoms are inflammatory heart disease, heart failure, dysrhythmia, heart attack, stroke, or abnormal clotting. Other symptoms of both long-haul and break-through covid-19 may include vasculitis, neuropsychiatric symptoms, conjunctivitis, and musculoskeletal pain. The inventive methods comprise the administration both acutely and chronically of pharmaceutical preparations comprising nitrone, nitroxide and nitroso compounds and their corresponding reduction products. Modes of administration include, but are not limited to, orally, intraorally, intranasally, topically to skin or mucosa, by nasal insufflation, inhalation, systemically by injection, or by local rectal administration.

Not applicable

Not applicable

### DETAILED DESCRIPTION OF INVENTION

### First Embodiment

Definitions

The terms “nitroxide”, “nitrone”, and “nitroso” are used herein to describe molecules comprising an oxygen and a nitrogen atom directly bound to each other. These compounds may be a electron donors or acceptors. Depending on their oxidation state, these compounds may comprise stable nitroxyl free radicals including precursors (such as the N—H form), and derivatives thereof including their corresponding hydroxylamine derivative (N—OH), where the oxygen atoms are replaced with a hydroxyl group and/or exist in a hydrogen halide form. Nitroxides and nitrones of the invention may be administered to a system, such as a human, and act to modulate oxidation and reduction reactions by donating or accepting an electron. Other mechanisms may include formation of charge-transfer complexes as well as by “redox signaling” or modulation of redox-signaling-mediated processes.

Stability of unpaired electrons on such compounds is typically-provided at the nitrogen nucleus by two adjacent carbon atoms that may be substituted with strong electron donor groups. With the partial negative charge on the oxygen of the N—O bond, the two adjacent carbon atoms together localize the unpaired electron on the nitrogen nucleus.

Nitroxides and nitrones generally may have either a heterocyclic or a linear structure. In an in vivo environment a nitroxide may react with a first superoxide to form oxoammonium (as an electron donor) and then react with a second superoxide to re-form the nitroxide (as an electron acceptor).

The terms “treat,” “treatment” and the like are used herein to generally mean obtaining a desired pharmacological and/or physiological effect in humans or other animals. A treatment is an approach for obtaining beneficial or desired clinical results. While the claims are not dependent on any specific mechanism, in the present case, these clinical results include but are not limited to decreasing undesirable effects of reactive oxygen species (ROS) and oxidative stress in general, as well as modulating more specific messenger processes such as “redox signaling”. The effect may be therapeutic in terms of a partial or complete cure of the disease and/or adverse effect attributed to the disease. In general, methods of the invention may be applied to a variety of different areas including the skin, mucus membranes including those in the GI tract, nose, throat, mouth, vaginal cavity, ocular surfaces, as well as the surfaces of the lungs and the surfaces of the vascular system as well as systemically by means of intravenous, intraocular, intramuscular, transdermal, sublingual, by insufflation, and/or intraoral administration. “Treatment” as used herein covers any treatment of such a symptom or disease in a mammal, particularly a human, and includes:

(I.a) inhibiting the disease, i.e. arresting it's development; or

(I.b) relieving the disease and/or it's symptom, i.e. causing regression of the disease and/or the symptoms caused by the disease.

Exemplary compounds include, but are not limited to, DEPMPO (5-(Diethoxyphosphoryl)-5-methyl-1-pyrroline N-oxide), TEMPO (2,2,6,6-tetramethyl-1-piperidinyl-1-oxyl), 4-Amino-TEMPO, 4-hydroxy-TEMPO (Tempol), DMPO (5,5-dimethylpyrroline-N-oxide), EMPO (2-Ethoxycarbonyl-2-methyl-3,4-dihydro-2H-pyrro-1-oxide), POBN (alpha-(4-pyridyl-1-oxide)-N-tert-butylnitrone), TEMPONE (4-Oxo-2,2,6,6-tetramethylpiperidine-1-oxyl 4-Oxo-TEMPO), TMIO 3,3,5,5 tetramethyl-1-pyrolline-N-oxide (TMPO), M3PO (2,5,5-trimethyl-1-pyrroline-N-oxide), M4PO (3,3,5,5-tetramethyl-1-pyrroline-N-oxide), TMPO (3,3,5,5 tetramethyl-1-pyrolline-N-oxide), PBN (1-alpha-phenyl-tert-butyl nitrone), Disulfenton (disulfonyl PBN), and MNP (2-methyl-2-nitrosopropane), and MitoTempol (2,2,6,6-Tetramethyl-4-[[5-(triphenylphosphonio)pentyl]oxy]-1-piperidinyl-oxy bromide), as well as their corresponding hydroxylamine derivatives and prodrugs (e.g., Zarling et al, 2015).

Such hydroxylamines act as prodrugs for the corresponding nitroxide and vice versa. In theory, providing the already reduced form may alleviate vitamin-c depletion when giving large amounts of the drug.

The various sulfone (e.g., NXY-059, disulfonyl PBN), hydroxyl, and other derivatives such as esters, peptides, hydroxyl, hydroxylamines, nitrones, carboxyls, and so forth are also claimed.

Preferred examples of the type of hydroxylamine compounds suitable for use in the present invention are Tempol-H (the hydroxylamine reduced form of the nitroxide 4-hydroxy-2,2,6,6-tetramethylpiperidin-1-yloxy), TEMPO-H (the hydroxylamine reduced form of the nitroxide 2,2,6,6-tetramethylpiperidin-1-yloxy) and OXANO-H (2-ethyl-2,4,4-trimethyloxazolidine, which is the reduced form of oxano, 2-ethyl-2,4,4-trimethyloxazolidin-3-yloxy), as well as MitoTempol-OH. Other hydroxylamine compounds suitable for use in the present invention include, but are not limited to, those disclosed by Hahn et al. (1998, supra; 2000, supra), Samuni et al. (2001, supra); and in U.S. Pat. No. 5,981,548 to Paolini, et al. (disclosing certain N-hydroxylpiperidine esters and their use as antioxidants in a number of contexts); U.S. Pat. No. 4,404,302 to Gupta et al. (disclosing the use of certain N-hydroxylamines as light stabilizers in plastics formulations); and U.S. Pat. No. 5,462,946 to Mitchell et al. (disclosing certain nitroxides deriving from substituted oxazolidines for protection of organisms from oxidative stress). Most of the above-referenced compounds have not been known heretofore for administration to humans. Certainly, none of them has been known for use in the treatment of respiratory coronavirus infection, much less Covid-19.

Suitable reducing agents include, but are not limited to: ascorbic acid, lipoic acid, cystine, purines and derivatives such as acetylcysteine, uric acid and other oxyxanthines, methionine, homocysteine, NADPH, and NADH.

### Operation—First Embodiment

1. Preparation of a Therapeutic Solution of Tempol-H

Tempol reacts stoichometrically with Vitamin-C to form Tempol-OH, which equilibrates with Tempol in vivo. That is, each compound is a prodrug for the other. So to prevent possible ascorbate depletion, it may be advantageously-administered as the hydroxylamine prodrug or along with ascorbate. Thus, Tempol-OH or an equivalent pharmacologically-active hydroxylamine is prepared by mixing together 6 grams of tempol or a pharmacologically effective amount of another spin-label or spin trap such as methynitrosopropane, 20 grams of ascorbic acid or other reducing compound in 100 ml of distilled water. The formulation is used as is. The solution is slightly-bitter. The formulation can be administered diluted in a suitable liquid such as juice, tea, or coffee. An equivalent dry form as 20 mg of Tempol mixed with 100 mg of ascorbic acid or equivalent reducing substance can be easily prepared in capsule or tablet form.

2. Treatment of Long-Haul Coronavirus Infection

A septuagenarian male experienced sudden frightening onset of severe headache, marked nausea and vomiting, pharyngitis, severe upper respiratory congestion, fever, chills, myalgia, weakness, malaise, and mild to moderate pulmonary congestion. O2 saturation=96%.

Patient had been taking Tempol off-and-on for years for cancer prophylaxis and for its possible antiaging activity, as well as some miscellaneous actions such as treatment of periodontitis and osteoarthritis. But, concerned that it would interfere with their activity, he had stopped Tempol about 6 weeks before receiving influenza (Fluvax) and Zoster (Zostavax) immunizations. The presumptive-diagnosis was “Covid-19”, possibly from a medically-related exposure.

Treatment: Nausea and vomiting continued without respite for several hours. Anti-nausea tablets were twice regurgitated intact.

Accordingly, treatment was initiated with 30 mg of Tempol PO as the hydroxylamine in a 6% water solution containing 90 mg vitamin-C. Patient kept this down. Along with Tempol-zinc gargles for pharyngitis, intranasal treatment was then initiated with a medication-soaked cotton swab and insufflation, repeated approximately q 6 h as symptoms returned. This was followed by similar doses of Tempol-H three and six hours later, repeated 2-3 times a day, PO for the next ten days. Subsequent intranasal treatment was also repeated 1-3 times a day for 10 days, as needed, titrating to relief of oropharyngeal symptoms as they recurred.

Results: On initiation of treatment, vomiting ceased almost immediately and did not recur. Within one to two hours, headache eased significantly, and the patient felt objectively better. Nasal congestion also quickly reversibly-eased upon repeated intranasal treatment. The speed of clinical response may signify some direct antiviral effect. Gargles provided relief from pharyngitis. Pulmonary congestion also cleared somewhat.

However, the patient experienced “vivid dreaming” that night and several subsequent ones, as well as overall “spaciness”. By days 5-7 remaining symptoms were mild reversible-on-treatment nasal congestion, pharyngitis, and malaise. Excepting slight malaise, patient was essentially asymptomatic by days 8-9. PCR for SARS-COV-2 RNA was negative at day 10. In retrospect, this may signify a direct antiviral effect of the application of Tempol directly to the upper respiratory mucosa and oropharynx by insufflation, inhalation, or gargling. Interestingly, although the diagnosis is reasonable secure, IGG antibodies against the virus were also negative at days 11 and 58, also arguably reflecting some direct antiviral activity.

3. Treatment of “Long-Haul” Covid-19

After patient had discontinued treatment above for several weeks after the first round of disease, subsequent putative recurrent post-acute (“long-haul”) symptoms of Covid-19. These included malaise, fatigue, brain fog, myalgia and musculoskeletal pain, and shortness of breath. These also responded reversibly to further Tempol treatment. The addition of periodic direct intranasal/lung inhalation of 0.1 ml of the 6% Tempol or Tempol-H solution administered 1-2 times/day titrated to a clinical response also proved helpful. Treatment was titrated to a clinical response, with a maximum dose of 200 mg/day of Tempol. Symptoms returned when the treatment was withdrawn, but responded to repeat treatment. At two such interludes, in place of the Tempol, patient took 100 mg of phenylbutylnitrone (PBN). This also produced some subjective clearing of symptoms. Simple nasal/pulmonary inhalation of a 0.1 ml of a 5% PBN solution in water 3-4 times per day also produced significant symptomatic relief.

4. Treatment of Long-Haul Covid-19-Induced Myalgia and Musculoskeletal Pain

About 38% of cases of long-haul covid-19 are associated with myalgia and musculoskeletal pain. Accordingly, a 2% hydroxycellulose gel containing 3% Tempol sufficient to cover the affected area was applied to the affected areas of the back and shoulders and rubbed in. Within a few minutes, significant local relief of myalgia and associated musculoskeletal pain was observed. This lasted about 3-4 hours. Further topical 3% Tempol gel as needed reproduced the analgesic effect. Occlusion of the applied gel by a shirt or by applying a water-impermiable plastic (“saran wrap”) covering improved the results. A 5% Tempol gel in a 1% hydroxycellulose gel was also effective but produced some skin irritation when used more than 3 days in a row.

Additional Embodiment: Treatment of “Break-Thru” Covid-19 Infection

Despite three doses of Pfizer vaccine, and intermittent Tempol treatment, months after the experience recounted above, patient developed “break-through” Covid-19. From the timing, this was almost certainly the Omicron strain. Treatment with 6% Tempol solution was initiated as described above, with the addition of tempol administered as 0.1 ml intranasal solution of 6% Tempol, from a intranasal spray bottle, as well as a daily oral dose of 100 mg of phenylbutylnitrone (PBN). Doses of Tempol were titrated to a clinical response, stopping and restarting treatment as indicted.

As an alternative, as symptoms recurred, variously Q-tip-mediated intranasal application of a 3% Tempol Gel or in the alternative, intranasal insufflation of 0.2 ml of a 3% solution or 0.1 ml of a 5% solution of phenylbutylnitrone (PBN) were used in place of the 6% Tempol nasal spray, again treating local symptoms as they recurred.

Results: Once again, all these treatments produced significant amelioration of symptoms. For example, alleviation of upper respiratory tract congestion upon inhalation of 20 mg of Tempol (one 0.1 ml “spritz” of the 6% solution per nostril, followed by insufflation) was fairly quick, taking only about 5-10 minutes. 3% Tempol gel or 3% phenylbutylnitrone solution administered as above were also effective but the response was slower.

Antiviral effectiveness of intranasal treatment is particularly significant because the Covid-19 virus first replicates in the upper respiratory tract, only later seeding the lungs and the rest of the body. So lowering viral titers at this early stage may help prevent more serious disease. There were no apparent side-effects from oral consumption. Intranasal use of all formulations was eventually associated with mild nasal irritation, which may have at least partially been due to the infection itself. The 3% gel is the least irritating.

Alternate forms of oral administration such as tablets, pills, or capsules are also effective. Parental modes of administration include intravenous, intramuscular, subcutaneous, intraperitoneal, transrectal, or by direct inhalation into lungs. Topical modes of administration include lotions, creams, gels, and topically-compatible suspensions and solutions. Tempol itself is effective at the same doses as Tempol-H, but may have increased side-effects at higher doses, e.g., due to oxidation of reducing agents or production of hydrogen peroxide. Such methods are routine in the art, and may vary with the needs of individual subjects.

The present invention is not limited to the embodiments described and exemplified above, but is capable of variation and modification within the scope of the appended claims. Also, the claims are not bound by any suggested possible mechanism of action and are independent thereof.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention. Likewise, each claim and indication stands independent of the patentability or patent status of any other claim and indication.

What is claimed is:

1. A method of treating a patient presenting with a non-respiratory symptom of a Long Covid infection, said method comprising administering as indicated during the course of the disease to a patient in need thereof of a therapeutically-effective amount of a compound selected from the group consisting of TEMPO (2,2,6,6-tetramethyl-1-piperidinyl-1-oxyl), TEMPONE,(4-oxo-2,2,6.6-tetramethylpiperidine-1-oxyl 4-oxo-TEMPO), TEMPOL (4-hydroxy-TEMPO), 4 amino-TEMPO, Mito-TEMPO (2-(2,2,6,6-Tetramethylpiperidin-1-oxyl-4-ylamino)-2-oxoethyl)triphenylphosphonium chloride), TEMPO-H (TEMPO-hydroxylamine), and TEMPOL-H (TEMPOL-hydroxylamine).

2. The method of claim 1, wherein the said compound is Tempol

3. The method of claim 1, wherein the patient's symptom to be treated comprises a dysfunction of the brain or nervous system

4. The method of claim 1, wherein the patient's symptom to be treated is brain fog

5. The method of claim 1, wherein the patient's symptom to be treated is post-exertional malaise

6. The method of claim 1, wherein the patient's symptom to be treated is cognitive dysfunction

7. The method of claim 1, wherein the patient's symptom to be treated is selected from the group comprised of memory deficit, hallucinations, anxiety, or depression

8. The method of claim 1, wherein the patient's symptom to be treated is selected from the group comprised of neuralgia, eye pain, bone pain, joint pain, conjunctivitis, speech impairment, headache, fatigue, neuropathic pain, tremor, malaise, weakness after exertion, muscle spasms, muscle aches, insomnia, sleep apnea, disorientation, inability to regulate temperature, musculoskeletal pain, or deficient bladder-control

9. The method of claim 1, wherein the patient's symptom to be treated is selected from the group comprised of ear pain, balance impairment, tinnitis, dizziness, vertigo, or deafness

10. The method of claim 1, where-in the patient's symptom to be treated is selected from the group comprised of heart palpitations, tachycardia, bradycardia, chest pain, vasculitis, thromboembolism, covid toe, myocarditis, myocardial infarction, or chest tightness

11. The method of claim 1, wherein a therapeutically-effective amount of the compound is administered as indicated during the course of the disease to a patient orally, sublingually, transrectally, topically to skin, to oropharyngeal mucosa, to conjunctival mucosa, by nasal insufflation, intramuscularly, intravenously, or subcutaneously, using clinically-appropriate formulations such as parenteral or topical solutions, dry powders, suspensions, gels, ointments, powders, or creams, and wherein such compound is administered at a dose comprising 0.01 mg/kg to 300 mg/kg
